(19) United States
(12) Patent Application Publication
(10) Pub. No.: US 2021/0225516 A1
(43) Pub. Date: Jul. 22, 2021

(54) METHOD AND DEVICE FOR PROCESSING PHYSIOLOGICAL DATA, AND STORAGE MEDIUM

(71) Applicants: BEIJING BOE HEALTH TECHNOLOGY CO., LD., Beijing (CN); BOE TECHNOLOGY GROUP CO., LTD., Beijing (CN)

(72) Inventors: Jinye LIU, Beijing (CN); Shuai CAO, Beijing (CN); Hui DU, Beijing (CN)

(73) Assignees: BEIJING BOE HEALTH TECHNOLOGY CO., LTD., Beijing (CN); BOE TECHNOLOGY GROUP CO., LTD., Beijing (CN)

(21) Appl. No.: 17/054,882

(22) PCT Filed: Mar. 19, 2020
(86) PCT No.: PCT/CN2020/080237

§ 371 Date: Nov. 12, 2020

(30) Foreign Application Priority Data
Apr. 24, 2019 (CN) 201910334395.1

(51) Int. Cl. G16H 50/30; A61B 5/0205; A61B 5/00; G16H 20/30

(52) U.S. Cl. CPC G16H 50/30; A61B 5/0205; A61B 5/02035; G16H 20/30; A61B 5/7225

(57) ABSTRACT

A method for processing physiological data, a device for processing physiological data, and a non-volatile storage medium are provided. The method includes: acquiring one or more physiological data of a detected object; calculating a state judgment value corresponding to each of the one or more physiological data based at least in part on a basic assignment table; and calculating health data of the detected object based at least in part on a weight assignment table and the state judgment value.

### CROSS-REFERENCE OF RELATED APPLICATIONS

The present application claims priority of Chinese patent application No. 201910334395.1, filed on Apr. 24, 2019, the entire disclosure of which is incorporated herein by reference as part of the present application.

### TECHNICAL FIELD

Embodiments of the present disclosure relate to a method for processing physiological data, a device for processing physiological data, and a storage medium.

### BACKGROUND

Currently, there are collection devices that can collect physiological data on the market, and these collection devices can acquire physiological data related to certain diseases. For example, professionals can take arterial blood and use an equipment related to pulmonary circulation to perform blood gas analysis to acquire physiological data related to pulmonary circulation. For another example, it is also possible to collect physiological data related to cardiovascular disease through an equipment related to cardiovascular disease.

Generally, these physiological data need to be processed manually to acquire the health data required by the detected object.

### SUMMARY

At least one embodiment of the present disclosure provides a method for processing physiological data, and the method comprises: acquiring one or more physiological data of a detected object, calculating a state judgment value corresponding to each of the one or more physiological data based at least in part on a basic assignment table, and calculating health data of the detected object based at least in part on a weight assignment table and the state judgment value.

At least one embodiment of the present disclosure provides a device for processing physiological data, and the device comprises an acquisition circuit which is configured to acquire one or more physiological data of a detected object, an area calculation circuit which is configured to calculate a state judgment value corresponding to each of the one or more physiological data based at least in part on a basic assignment table, and a comprehensive calculation circuit which is configured to calculate health data of the detected object based at least in part on a weight assignment table and the state judgment value.

At least one embodiment of the present disclosure provides a device for processing physiological data, the device comprises a processor and a memory, the memory stores computer executable instructions, and the computer executable instructions are executed by the processor to perform the method for processing physiological data according to any one of the embodiments of the present disclosure.

At least one embodiment of the present disclosure provides a non-volatile storage medium, which stores computer executable instructions, and the computer executable instructions are executed by a processor to perform the method for processing physiological data according to any one of the embodiments of the present disclosure.

### BRIEF DESCRIPTION OF THE DRAWINGS

In order to clearly illustrate the technical solution of the embodiments of the present disclosure, the drawings of the embodiments will be briefly described in the following. It is obvious that the described drawings in the following are only related to some embodiments of the present disclosure and thus are not limitative of the present disclosure.

FIG. 1 is a flow diagram of a method for processing physiological data provided by at least one embodiment of the present disclosure;

FIG. 2 is a schematic diagram of a method for processing physiological data provided by at least one embodiment of the present disclosure;

FIG. 3 is another flow diagram of a method for processing physiological data provided by at least one embodiment of the present disclosure;

FIG. 4A is another schematic diagram of a method for processing physiological data provided by at least one embodiment of the present disclosure;

FIG. 4B is further another schematic diagram of a method for processing physiological data provided by at least one embodiment of the present disclosure;

FIG. 5A is a structural diagram of a device for processing physiological data provided by at least one embodiment of the present disclosure;

FIG. 5B is another structural diagram of a device for processing physiological data provided by at least one embodiment of the present disclosure;

FIG. 6 is a block diagram of a device for processing physiological data provided by at least one embodiment of the present disclosure; and

FIG. 7 is a structural diagram of a computing system for realizing the method provided by at least one embodiment of the present disclosure.

### DETAILED DESCRIPTION

In order to make objects, technical details and advantages of the embodiments of the disclosure apparent, the technical solutions of the embodiment will be described in a clearly and fully understandable way in connection with the drawings related to the embodiments of the disclosure. It is apparent that the described embodiments are just a part but not all of the embodiments of the disclosure. Based on the described embodiments herein, those skilled in the art may obtain other embodiment, without any creative work, which shall be within the scope of the disclosure.

It should be noted that in the present specification and drawings, steps and elements, which are the same or similar, are denoted by same or similar reference numerals, and repeated descriptions of these steps and elements will be omitted.

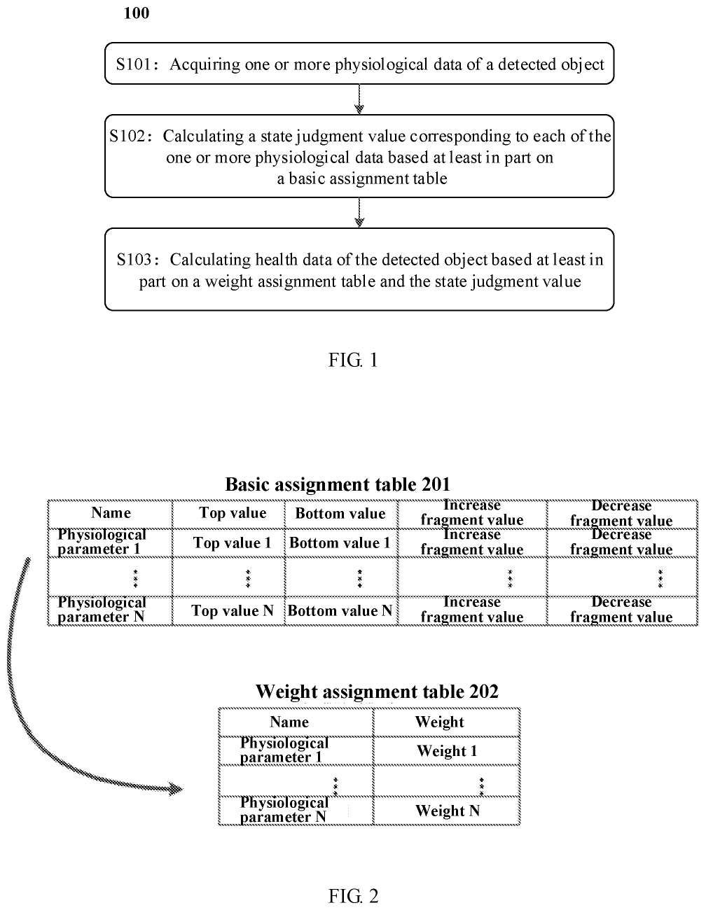

FIG. 1 is a flow diagram of a method 100 for processing physiological data provided by at least one embodiment of the present disclosure.

Referring to FIG. 1, the method 100 for processing physiological data provided by at least one embodiment of the present disclosure includes the following steps S101-S103.

Step S101: acquiring one or more physiological data of a detected object.

Step S102: calculating a state judgment value corresponding to each of the one or more physiological data based at least in part on a basic assignment table.

Step S103: calculating health data of the detected object based at least in part on a weight assignment table and the state judgment value.

The above physiological data may be, for example, various data that are associated with the health state of the detected object and can be directly detected by a vital sign detection device. For example, the data may be height, weight, body fat, one or more physiological data related to the working state of the cardiovascular system, one or more physiological data related to the working state of the pulmonary circulation function, one or more physiological data related to the working state of the stomach and intestines, and the like. Normally, these physiological data are related to the health state of the detected object to a certain extent.

The above health data may be, for example, various data associated with the health state of the detected object. These data cannot be directly detected by the vital sign detection device. For example, in the case where the detected physiological data are physiological data such as height, weight, body fat, etc., the health data may involve the health data of whether the detected object is overweight, whether the detected object is too thin, or whether the detected object needs to strengthen exercise, etc. In the case where the detected physiological data is one or more physiological data related to the working state of the cardiovascular system, the health data may be data for evaluating whether the working state of the cardiovascular system of the detected object is normal, or for determining whether the detected object may be at risk of cardiovascular disease, or for providing information on whether the detected object needs to be supplemented with nutrients related to cardiovascular health, etc.

Similarly, the health data related to the working states of the pulmonary circulation function and/or the stomach and intestines may be data for evaluating whether the working states of the pulmonary circulation function and/or the stomach and intestines of the detected object are normal, or for determining whether the detected object may be at risk of pulmonary circulation function and/or stomach and intestines diseases, or for providing information on whether the detected object needs to be supplemented with nutrients related to the pulmonary circulation function and/or the stomach and intestines health.

With the method 100 of at least one embodiment of the present disclosure, the acquired physiological data can be used to obtain the health data of the detected object in a home environment without the need for a professional to manually process the physiological data of the detected object. For example, a basic assignment table can be used to calculate whether the detected physiological data is in the normal threshold range by a general-purpose computer, a special-purpose computer, or the like. The above basic assignment table can include thresholds related to various physiological data. The state judgment value corresponding to each of the one or more physiological data can be used to indicate whether the physiological data is in the normal threshold range. In the case where a certain physiological data is too far from the normal threshold range, the state judgment value corresponding to the certain physiological data can change drastically to represent the abnormal state of the physiological data.

After acquiring the state judgment value corresponding to each physiological data, a computer device can also be used to comprehensively calculate the above health data based on a weight assignment table. For example, when it is necessary to calculate the health data related to the working state of the cardiovascular system, the weight of the state judgment value corresponding to the physiological data associated with the stomach and intestines may be lower, while the weight of the state judgment value corresponding to the physiological data related to the working state of the cardiovascular system may be higher. As a result, accurate and effective health data related to the working state of the cardiovascular system can be obtained, and the process of acquiring health data related to the working state of the cardiovascular system can be simplified.

The above one or more physiological data can be acquired by a non-invasive vital sign detection device. The non-invasive vital sign detection device can includes, but is not limited to, a wearable electronic equipment, a monitor, a sports bracelet, a fingertip measuring instrument, a home portable blood pressure monitor, a home portable breathing detector, a smart body fat measuring instrument, a smart weight measuring instrument, etc. The non-invasive vital sign detection device can also include one or more of a motion sensor such as a gyroscope, an acceleration sensor, etc., a temperature sensor, a humidity sensor, a central processing unit (CPU), a memory, an image processing unit (GPU), a display screen, a communication device, a wireless network card, and the like.

The method 100 for processing physiological data provided by at least one embodiment of the present disclosure can be executed by, for example, a central processing unit or an image processing unit for the non-invasive vital sign detection device in cooperation with corresponding computer program codes. The non-invasive vital sign detection device can further adopt various appropriate operating systems, such as an Android system, a Linux system, a Unix system, or a Windows system, etc., the embodiments of the present disclosure are not limited in this aspect.

The method 100 for processing physiological data provided by at least one embodiment of the present disclosure, for example, can also be executed by one or more electronic devices that are wired or wirelessly connected to the non-invasive vital sign detection device. These electronic devices include, but are not limited to, a mobile phone, a personal computer, a tablet computer, a workstation, a cloud server, etc.

The above non-invasive vital sign detection device can replace the invasive vital sign detection device that needs to acquire physiological data by professionals in the hospital through invasive methods, thereby achieving the technical effect of simply and conveniently acquiring and processing relevant physiological data.

FIG. 2 is a schematic diagram of a method 100 for processing physiological data provided by at least one embodiment of the present disclosure.

Referring to FIG. 2, the basic assignment table in the method 100 of at least one embodiment of the present disclosure can be the basic assignment table 201 in FIG. 2. The basic assignment table 201 can include at least one of following assignment items corresponding to the one or more physiological data: a top value, a bottom value, an increase fragment value, and a decrease fragment value. The increase fragment value indicates the influence of the difference between the value of a physiological parameter and the top value on the state judgment value in the case where the value of the physiological parameter exceeds the top value. Similarly, the decrease fragment value indicates the influence of the difference between the value of the physiological parameter and the bottom value on the state judgment value in the case where the value of the physiological parameter is less than the bottom value.

For example, corresponding to a certain physiological data such as a physiological parameter N (that is, the N-th physiological parameter, in which N is an integer greater than or equal to 1), the basic assignment table 201 can include a top value RC_{N}, a bottom value RF_{N}, an increase fragment value α_{N} and a decrease fragment value β_{N}, etc.

The value ranges of the top value RC_{N} and the bottom value RF_{N} can be determined based on the physiological parameter N. For example, in the case where the physiological parameter N represents height, the top value RC_{N} can be the height of an ordinary person with a higher height (for example, 2 meters), and the bottom value RF_{N} can be the height of an ordinary person with a lower height (for example, 1.5 meters). Similarly, the increase fragment value α_{N} and the decrease fragment value β_{N} can also be determined based on the physiological parameter N. For example, the increase fragment value α_{N} and the decrease fragment value β_{N} corresponding to the physiological parameter N representing the height can be, for example, any value between 0.01-0.1 meters. The increase fragment value α_{N} and the decrease fragment value β_{N} may be the same or different. For another example, in the case where the physiological parameter N represents the systolic blood pressure, the bottom value RF_{N} can be 120 mmHg, and the top value can be 139 mmHg. In the case where the physiological parameter N represents the diastolic blood pressure, the value of the diastolic blood pressure is 80 to 89 mmHg, the increase fragment value α_{N} can be set to 20 mmHg, and the decrease fragment value β_{N} can be set to 10 mmHg.

Optionally, in some examples, the top value RC_{N} and the bottom value RF_{N} are used to respectively indicate the maximum value and the minimum value that the physiological parameter N can have in the case where the physiological parameter N belongs to the normal threshold range. The increase fragment value α_{N} and the decrease fragment value β_{N} are used to respectively indicate the influence of the deviation value of the physiological parameter N from the normal threshold range on the state judgment value in the case where the physiological parameter N does not belong to the normal threshold range. For example, the increase fragment value α_{N} can be used to indicate the influence of the deviation value (for example, the difference between the value of the physiological parameter N and the top value RC_{N}) on the state judgment value in the case where the value of the physiological parameter N is greater than the top value RC_{N}. When the increase fragment value α_{N} is larger, it means that the influence of the deviation value on the state judgment value is smaller in the case where the value of the physiological parameter N is greater than the top value RC_{N}. Similarly, the decrease fragment value β_{N} can be used to indicate that the influence of the deviation value (for example, the difference between the bottom value RF_{N} and the value of the physiological parameter N) on the state judgment value in the case where the value of the physiological parameter N is less than the bottom value RF_{N}. When the decrease fragment value β_{N} is larger, it means that the influence of the deviation value on the state judgment value is smaller in the case where the value of the physiological parameter N is smaller than the bottom value RF_{N}.

With continued reference to FIG. 2, the weight assignment table in the method 100 of at least one embodiment of the present disclosure can be the weight assignment table 202 in FIG. 2. The weight assignment table 202 can include weight values corresponding to one or more physiological data.

For example, corresponding to a certain physiological data (for example, the physiological parameter N), the weight assignment table 202 can include a weight ω_{N}.

Optionally, the health data of the detected object can be acquired by calculating the weighted average value based on the state judgment value of one or more physiological data and the weight value of one or more physiological data. The weight value of a certain physiological data is related to the degree of the correlation between the certain physiological data and the health state.

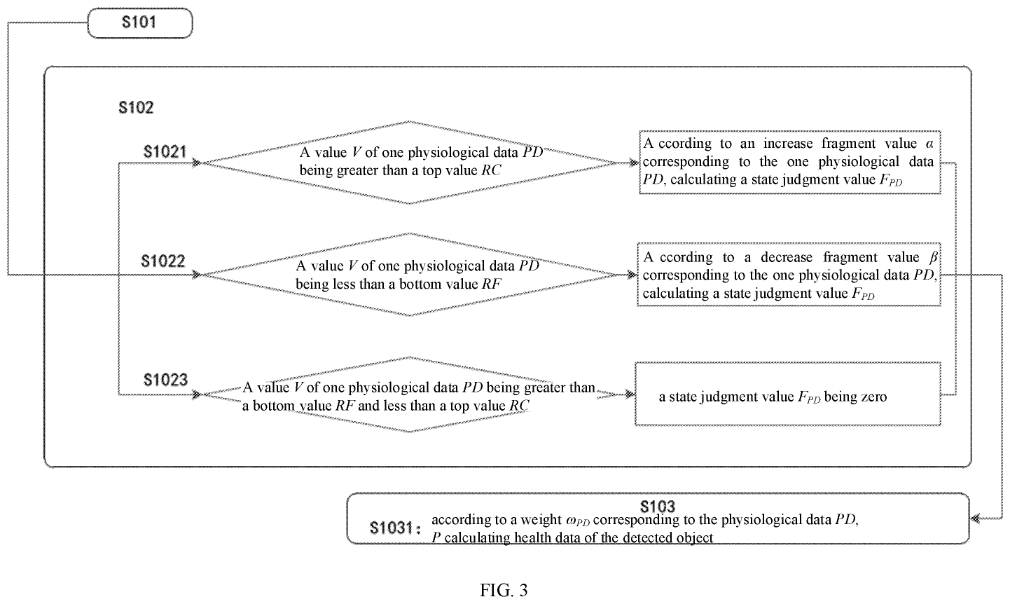

The method 100 for processing physiological data provided by at least one embodiment of the present disclosure is further described below with reference to FIG. 3.

FIG. 3 is another flow diagram of a method 100 for processing physiological data provided by at least one embodiment of the present disclosure.

Referring to FIG. 3, the step S102 of the method 100 for processing physiological data provided by at least one embodiment of the present disclosure includes the following steps S1021 to S1023 according to different situations.

Step S1021: in response to a value V of one physiological data PD of the one or more physiological data being greater than a top value RC corresponding to the one physiological data PD, and according to an increase fragment value α corresponding to the one physiological data PD, calculating a state judgment value F_{PD} corresponding to the one physiological data PD, in which

$${F}_{PD} = 1 - {2}^{\frac{V - RC}{α}} .$$

Step S1022: in response to a value V of one physiological data PD of the one or more physiological data being less than a bottom value RF corresponding to the one physiological data PD, and according to a decrease fragment value β corresponding to the one physiological data PD, calculating a state judgment value F_{PD} corresponding to the one physiological data PD, in which

$${F}_{P D} = 1 - {2}^{\frac{R F - V}{β}} .$$

Step S1023: in response to a value V of one physiological data PD of the one or more physiological data being greater than a bottom value RF corresponding to the one physiological data PD and less than a top value RC corresponding to the one physiological data PD, a state judgment value F_{PD} corresponding to the one physiological data PD is zero.

Therefore, the above steps S1021 to S1023 can at least realize that in the case where the value V of the physiological data PD is in the normal threshold range (for example, is greater than the bottom value RF and less than the top value RC), the state judgment value is a stable value (for example, zero) to indicate that the physiological data of the detected object is normal data, and in the case where the value V of the physiological data PD is not in the normal threshold range (for example, is less than the bottom value RF or greater than the top value RC), the state judgment value changes drastically to indicate the abnormality of the physiological data.

According to the above steps S1021 to S1023, in the case where the value V of the physiological data PD deviates farther from the normal data, the state judgment value is negative and the change is more severe. For example, generally, in the case where a certain physiological data only slightly exceeds the top value, it is usually judged as slight abnormality, and the influence on the health data of the detected object is also small. In the case where a certain physiological data greatly exceeds the top value, it is necessary to prompt the detected object that the physiological data is seriously abnormal. Therefore, the state judgment value in this case needs to be a value that is significantly different from the stable value in the normal state, which reflects that the physiological data has a greater influence on the health data. Therefore, the abnormality degree of the physiological data can be more intuitively evaluated in this way.

According to the above steps S1021 to S1023, the larger the increase fragment value α or the decrease fragment value β is, the smaller the change of the state judgment value is. Therefore, the degree of change of the state judgment value can be adjusted by the increase fragment value α or the decrease fragment value β. For example, in the case where the value of a certain physiological data has a larger order of magnitude, the increase fragment value α and the decrease fragment value β can also be larger, so that the acquired state judgment value can more accurately measure the abnormality degree of the physiological data.

The step S103 of the method 100 for processing physiological data provided by at least one embodiment of the present disclosure can include the following step S1031.

Step S1031: according to a weight ω_{PD} corresponding to each of the one or more physiological data in the weight assignment table, calculating the health data P of the detected object, in which P=S+Σ_{PD=1}ⁿF_{PD}*ω_{PD}, n represents a count of the one or more physiological data, F_{PD} represents a state judgment value corresponding to the physiological data PD, and S represents a maximum value of the health data of the detected object.

According to the above step S1031, for example, in the case where the state judgment value is zero, it indicates that the physiological data is in the normal threshold range and does not affect the health data; in general, in the case where the physiological data is outside the normal threshold range, the state judgment value is a negative value; in the case where the absolute value of the state judgment value is larger, it indicates that the physiological data is outside the normal threshold range and may have a great influence on the health data.

The above health data P can be used to represent the influence of a combination of multiple physiological data on the health data. In the case where the health data is closer to S, it indicates that the detected object is in a healthier state.

Therefore, the health data of the detected object can be conveniently calculated by a computer device through the above steps S1021 to S1023 and step S1031 without the need for manual processing of the physiological data.

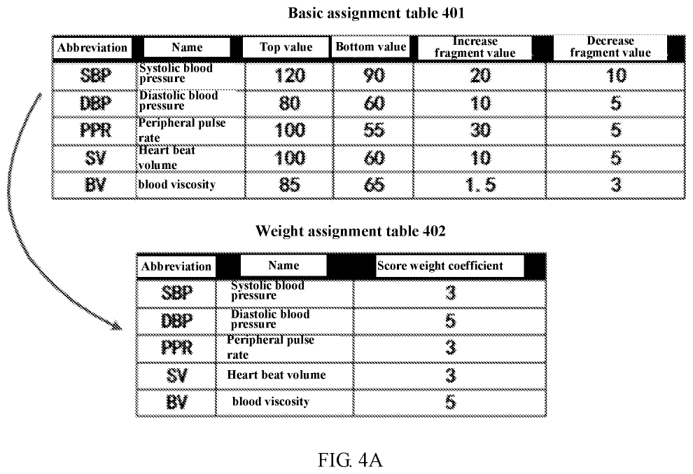

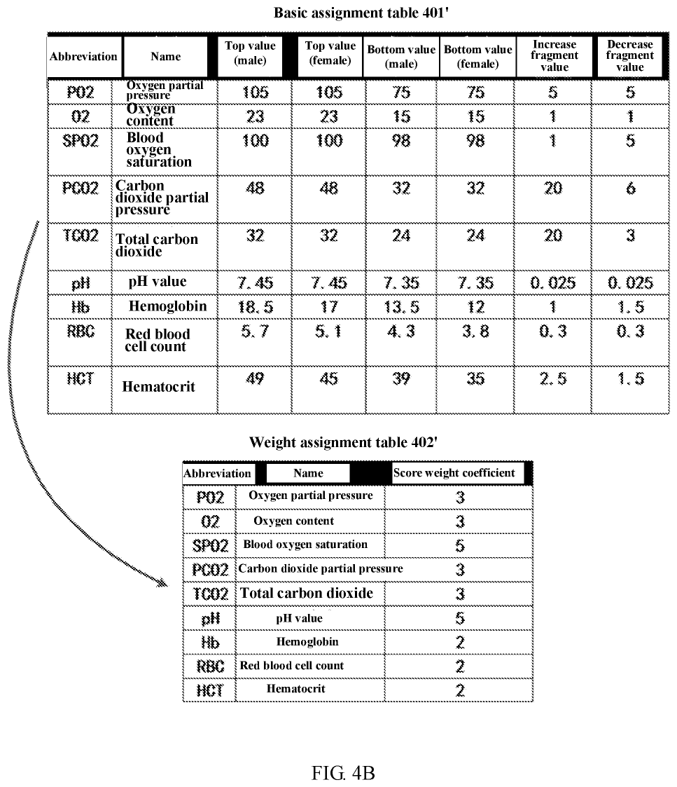

The method 100 for processing physiological data provided by at least one embodiment of the present disclosure is further described below with reference to FIG. 4A and FIG. 4B.

FIG. 4A is another schematic diagram of a method for processing physiological data provided by at least one embodiment of the present disclosure.

In the method 100 of at least one embodiment of the present disclosure, in response to the health data of the detected object being associated with the cardiovascular state of the detected object, the one or more physiological data include at least one of the following items: systolic blood pressure, diastolic blood pressure, peripheral pulse rate, heart beat volume, and blood viscosity.

According to the physiological data associated with the cardiovascular state, health data related to the working state of the cardiovascular system can be conveniently acquired.

Cardiovascular disease is a disease that endangers the health and life of the detected object, and has high mortality and high disability rate. At present, the prevalence of cardiovascular disease is continuously rising. The mortality rate of cardiovascular disease is still the first, higher than that of tumors and other diseases. Although the pathogenesis of cardiovascular disease remains to be studied, most of the physiological parameters that evaluate the health of the cardiovascular system are known, such as one or more of the above systolic blood pressure, diastolic blood pressure, peripheral pulse rate, heart beat volume, and blood viscosity.

The cardiovascular health data can be acquired based on the values of physiological parameters associated with cardiovascular disease and combining these values. The cardiovascular health data can reflect the cardiovascular health state or the risk of cardiovascular disease, so as to consider the prevention and control of cardiovascular disease.

Generally, for a certain cardiovascular disease, professionals can measure the risk of a detected object for the certain cardiovascular disease. In this case, professionals need to use an invasive device to detect physiological data, that is, a professional device is needed to collect physiological signals of the detected object, which is not convenient for real-time monitoring at home. According to at least one embodiment of the present disclosure, a non-invasive vital sign detection device can be used to detect physiological data related to cardiovascular health state anytime and anywhere. The method according to at least one embodiment of the present disclosure processes physiological data such as blood pressure, pulse rate, heart beat volume, and blood viscosity continuously detected by the non-invasive vital sign detection device to calculate a state judgment value, and then combines the weight corresponding to each physiological data to comprehensively calculate the cardiovascular health data, so as to achieve the effect of simplifying the evaluation process and facilitating operation, thus solving the technical problem of acquiring cardiovascular health data at any time and any place only by knowing the physiological data related to cardiovascular disease.

FIG. 4B is further another schematic diagram of a method for processing physiological data provided by at least one embodiment of the present disclosure;

In the method 100 of at least one embodiment of the present disclosure, in response to the health data of the detected object being associated with a pulmonary circulation function state of the detected object, the one or more physiological data include at least one of the following items: oxygen partial pressure, oxygen content, blood oxygen saturation, carbon dioxide partial pressure, total carbon dioxide, pH value, hemoglobin, red blood cell count, and hematocrit.

According to the above physiological data associated with the pulmonary circulation function state, the health data related to the pulmonary circulation function state can be conveniently acquired.

In general cases, the blood circulation is a dual circulation including systemic circulation and pulmonary circulation. When the ventricle contracts, blood enters the pulmonary artery from the right ventricle, and reaches the pulmonary capillaries through its branches, where gas exchange takes place, and venous blood becomes arterial blood. The pulmonary circulation has several characteristics: the path of the pulmonary circulation is shorter than the path of the systemic circulation, the vessel wall is thin, and the pressure is low; the lung tissue and pulmonary blood vessels are easy to expand, and the blood volume is larger; the pulmonary circulation is innervated by the sympathetic nerve and the vagus nerve; and when the oxygen partial pressure of part of the alveoli is decreased because of hypoventilation, the blood vessels around the part of alveoli constrict and the blood flow decreases, which allows more blood to flow through the alveoli with adequate ventilation and high oxygen partial pressure. The pulmonary circulation function is responsible for the body to take in oxygen and remove carbon dioxide, so as to change the corresponding components in the blood.

It is usually necessary for professionals to measure physiological data such as oxygen partial pressure, carbon dioxide partial pressure, blood oxygen saturation in the blood using a professional equipment for blood gas analysis to acquire health data related to pulmonary circulatory function, which is not convenient for home use. According to at least one embodiment of the present disclosure, the non-invasive vital sign detection device can be used to detect health data related to the health of the pulmonary circulation function anytime and anywhere. The method provided by at least one embodiment of the present disclosure processes the oxygen partial pressure, oxygen content, blood oxygen saturation, carbon dioxide partial pressure, total carbon dioxide, pH value, hemoglobin, red blood cell count, and hematocrit continuously detected by the non-invasive vital sign detection device to calculate the state judgment value, and then combines the corresponding weight of each physiological data to comprehensively calculate the health data related to the pulmonary circulation function, so as to achieve the technical effect of simplifying the evaluation process and facilitating operation. The health data evaluates the blood circulation state and the ability to carry oxygen and carbon dioxide through continuously monitoring of the relevant parameters of the pulmonary circulation, thereby comprehensively analyzing the health state of the pulmonary circulation function through a non-invasive method.

Optionally, any one of the top value and the bottom value in the method 100 for processing physiological data provided by at least one embodiment of the present disclosure can be associated with the gender of the detected object. In addition, calculating the state judgment value corresponding to each of the one or more physiological data includes: acquiring a bottom value and a top value of an assignment item corresponding to each of the one or more physiological data according to the gender of the detected object.

Therefore, more accurate health data can be acquired based on the gender of the detected object.

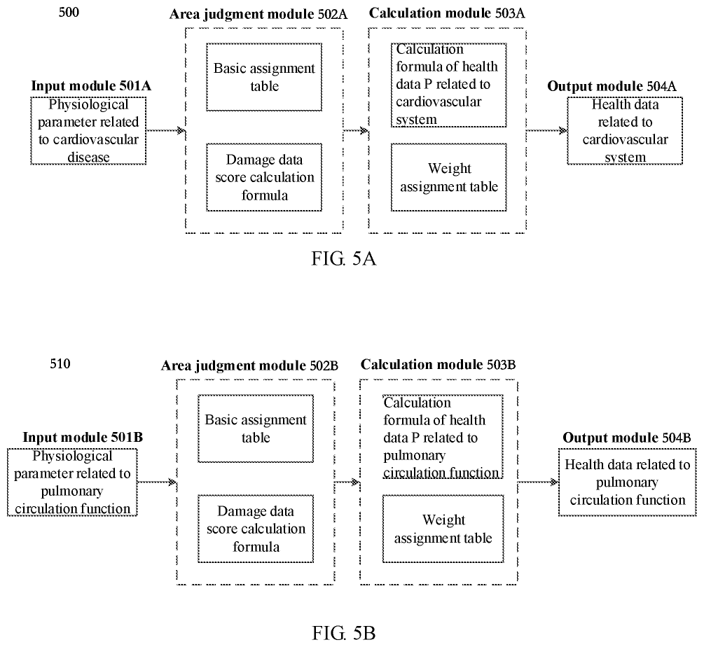

A device for processing physiological data provided by at least one embodiment of the present disclosure is described below with reference to FIG. 5A and FIG. 5B.

FIG. 5A is a structural diagram of a device 500 for processing physiological data provided by at least one embodiment of the present disclosure.

Referring to FIG. 5A, the device 500 for processing physiological data provided by at least one embodiment of the present disclosure can be used to evaluate cardiovascular health state.

Optionally, the device 500 for processing physiological data provided by at least one embodiment of the present disclosure includes the following four modules to process physiological data: an input module 501A, an area judgment module 502A, a calculation module 503A, and an output module 504A.

Optionally, the device 500 for processing physiological data provided by at least one embodiment of the present disclosure can acquire a physiological parameter V related to cardiovascular disease through the input module 501A, and then calculate a state judgment value F by using the damage data score calculation formula in the area judgment module 502A and referring to the basic assignment table. Then, the calculation module 503A is used to multiply the state judgment value F and the corresponding health data weight to obtain damage data deduction items, the multiple damage data deduction items are accumulated to obtain a total deduction item, and the total score (for example, 100 points) subtracts the total deduction item to obtain the cardiovascular health data. Finally, the output module 504A can be used to output the obtained cardiovascular health data.

Optionally, the data input in the input module 501A can be derived from the physiological parameters related to cardiovascular disease collected by the non-invasive vital sign detection device, for example, the physiological parameters include blood pressure (systolic blood pressure, diastolic blood pressure), peripheral pulse rate, heart beat volume, and blood viscosity.

Optionally, the damage data score calculation formula in the area judgment module 502A is as follows:

$$F = \{ \begin{matrix}1 - {2}^{\frac{V - RC}{α}} , & if V > RC & \\ 1 - {2}^{\frac{RF - V}{β}} , & if V < RF & \\ 0 , & if RF \leq V \leq RC & \end{matrix}$$

In the above formula, the F value represents a state judgment value of a certain physiological data V related to the cardiovascular system, and the F value increases sharply as the deviation of the physiological data V from the normal threshold range increases. In the case where the physiological data V is greater than the top value RC, formula \textcircled{1} can be used. In the case where the physiological data V is less than the bottom value RF, formula \textcircled{2} can be used. In the case where the physiological data V is not in the normal threshold range, the F value is less than zero. In the case where the physiological data V is in the normal threshold range, the formula \textcircled{3} can be used, and in this case, the F value is 0.

Referring again to FIG. 4A, the basic assignment table in the area judgment module 502A can be the basic assignment table 401 shown in FIG. 4A.

The RF value to RC value can be represented by the normal threshold range of various health data in the medical standard guide, for example, the normal threshold range of blood pressure can be 90-120 mm/Hg.

The increase fragment value α and the decrease fragment value β can be set hierarchically according to the severity of each health data. The increase fragment value α represents the increase degree of the F value.

For example, when the detected object is in a healthy state, the blood pressure of the detected object is usually less than 120/80 mmHg. According to the medical standard guide, when the detected object has borderline hypertension, the systolic blood pressure of the detected object is usually 120 to 139 mmHg, and the diastolic blood pressure is 80 to 89 mmHg. When the detected object has grade 1 hypertension, the systolic blood pressure of the detected object is usually 140 to 159 mmHg, the diastolic blood pressure is 90 to 99 mmHg, and so on. Therefore, the increase fragment value α of systolic blood pressure can be set to 20 mmHg and the decrease fragment value β of diastolic blood pressure can be set to 10 mmHg.

The state judgment value corresponding to the physiological data V can be obtained by the damage data score calculation formula in the area judgment module 502A.

Optionally, the module 503A can include the weight assignment table 402 in FIG. 4A. The health data related to cardiovascular system in module 503A can be: P=100+ΣFᵢ*ωᵢ. Fᵢ and ωᵢ are respectively the score and the weight of the corresponding physiological data V. The setting of the weight value is related to the degree of correlation between the physiological data V and the cardiovascular health state.

FIG. 5B is another structural diagram of a device for processing physiological data provided by at least one embodiment of the present disclosure.

Referring to FIG. 5B, the device 510 for processing physiological data provided by at least one embodiment of the present disclosure can also be used to evaluate the state of pulmonary circulation function.

The device 510 for processing physiological data provided by at least one embodiment of the present disclosure can use the following four modules to process physiological data: an input module 501B, an area judgment module 502B, a calculation module 503B, and an output module 504B.

Optionally, the device 510 for processing physiological data provided by at least one embodiment of the present disclosure can acquire a physiological parameter V related to pulmonary circulation function disease through the input module 501A, and then calculate a state judgment value F by using the damage data score calculation formula in the area judgment module 502B and referring to the basic assignment table. Then, the calculation module 503B is used to multiply the state judgment value F and the corresponding health data weight to obtain damage data deduction items, and the multiple damage data deduction items are accumulated to obtain a total deduction item. The total score (for example, 100 points) subtracts the total deduction item to obtain the health data according to the pulmonary circulation function. Finally, the output module 504B can be used to output the obtained health data according to the pulmonary circulation function.

Optionally, the data input in the input module 501B can be derived from the physiological parameters related to pulmonary circulation function collected by the non-invasive vital sign detection device, for example, the physiological parameters include oxygen partial pressure, oxygen content, blood oxygen saturation, carbon dioxide partial pressure, total carbon dioxide, pH value, hemoglobin, red blood cell count, and hematocrit.

Optionally, the damage data score calculation formula in the area judgment module 502B can be similar to the above formula \textcircled{1} to \textcircled{3}.

Referring again to FIG. 4B, the basic assignment table in the area judgment module 502B can be the basic assignment table 401′ shown in FIG. 4B. The RF value to RC value can be represented by the normal threshold range of various health data in the medical standard guide, for example, the normal threshold range of blood oxygen saturation can be 98%-100%.

Similarly, the health data related to the pulmonary circulation function of the detected object can be obtained by incorporating the damage health data Fᵢ and the weight coefficient co, related to the pulmonary circulation function into the formula: P=100+ΣFᵢ*ωᵢ.

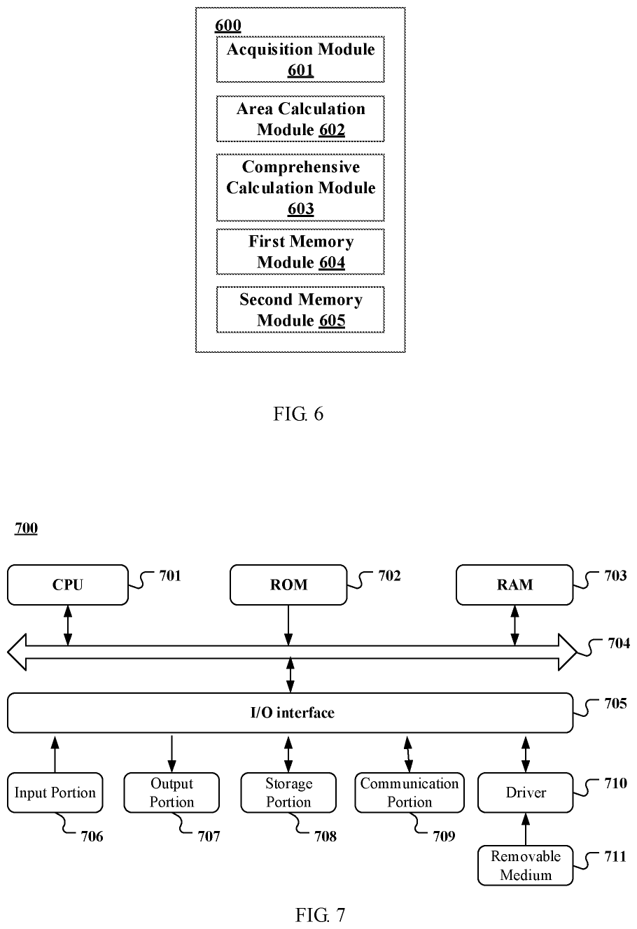

The device 600 for processing physiological data provided by at least one embodiment of the present disclosure is described below with reference to FIG. 6.

FIG. 6 is a block diagram of a device for processing physiological data provided by at least one embodiment of the present disclosure. The device 600 for processing physiological data includes an acquisition module 601, an area calculation module 602, a comprehensive calculation module 603, a first memory module 604 and a second memory module 605.

Optionally, the acquisition module 601 is configured to acquire one or more physiological data of a detected object.

Optionally, the area calculation module 602 is configured to calculate a state judgment value corresponding to each of the one or more physiological data based at least in part on a basic assignment table.

Optionally, the comprehensive calculation module 603 is configured to calculate health data of the detected object based at least in part on a weight assignment table and the state judgment value.

The above acquisition module 601, the area calculation module 602, and the comprehensive calculation module 603 can be implemented by software, hardware and firmware or any combination thereof, for example, implemented as an acquisition circuit, an area calculation circuit, and a comprehensive calculation circuit, respectively. The method steps of other embodiments of the present disclosure can also be implemented by software, hardware and firmware or any combination thereof in the same way.

Optionally, the first memory module 604 stores the basic assignment table in an appropriate form (such as a data table, a data file, a database, etc.). The second memory module 605 stores the weight assignment table in an appropriate form. For example, the first memory module 604 and the second memory module 605 are a first memory and a second memory, respectively.

The device provided by the embodiments of the present disclosure can include, but is not limited to, a smart phone, a tablet computer, a media player, and the like. It should be noted that for the sake of clarity, the entire structure of the device is not provided. In order to realize the necessary functions of the device, those skilled in the art can set other structures not shown according to specific application scenarios, and the present disclosure is not limited in this aspect.

In addition, at least one embodiment of the present disclosure further provides a device for processing physiological data, which includes a processor and a memory, the memory stores computer executable instructions, and the computer executable instructions are executed by the processor to perform the method for processing physiological data according to any one of the above embodiments.

In addition, at least one embodiment of the present disclosure further provides a non-volatile storage medium, which stores computer executable instructions, and the computer executable instructions are executed by a processor to perform the method for processing physiological data according to any one of the above embodiments.

The method for processing physiological data, the device for processing physiological data, and the non-volatile storage medium of the embodiments of the present disclosure can simplify the evaluation process of health data and facilitate the operation of the detected object, thereby solving the technical problem of comprehensively analyzing the physiological data of the detected object in a non-invasive manner.

The computer-readable storage medium can be one computer-readable storage medium or any combination of more computer-readable storage media. For example, one computer-readable storage medium stores computer-readable program codes for randomly generating a sequence of instructions for calculating a state judgment value corresponding to each of the one or more physiological data, and another computer-readable program medium stores computer-readable program codes for calculating the health data of the detected object.

Referring to FIG. 7, FIG. 7 is a structural diagram of a computing system 700 for realizing the method provided by at least one embodiment of the present disclosure.

As shown in FIG. 7, the computing system 700 includes a central processing unit (CPU) 701, which can perform various appropriate actions and processing according to the program stored in a read-only memory (ROM) 702 or the program loaded from a storage portion 708 into a random access memory (RAM) 703. In the RAM 703, various programs and data required for the operation of the computing system 700 are also stored. The CPU 701, ROM 702, and RAM 703 are connected to each other through a bus 704. An input/output (I/O) interface 705 is also connected to the bus 704.

The following components are connected to the I/O interface 705: an input portion 706 including a keyboard, a mouse, and an imaging device such as a camera; an output portion 707 including such as a cathode ray tube display screen, a liquid crystal display screen, and a speaker, etc.; a storage portion 708 including a hard disk; and a communication portion 709 including a network interface card such as a LAN card, a modem, etc. The communication portion 709 performs communication processing via a network such as the Internet. A driver 710 is also connected to the I/O interface 705 as required. A removable medium 711, such as a magnetic disk, an optical disk, a magneto-optical disk, a semiconductor memory, etc., is installed on the driver 710 as required, so that the computer program read therefrom is installed into the storage portion 708 as required.

In particular, according to the embodiments of the present disclosure, the process described above can be implemented as a computer software program. For example, an embodiment of the present disclosure provides a computer program product, which includes a computer program tangibly embodied on a machine-readable medium, and the computer program includes program codes for executing the method of the above process. In such an embodiment, the computer program may be downloaded from the network through the communication portion 709 and installed, and/or installed from the removable medium 711.

The flow diagrams and block diagrams in the accompanying drawings illustrate the possible implementation architecture, functions, and operations of the system, method, and computer program product according to various embodiments of the present disclosure. In this regard, each block in the flow diagrams or block diagrams can represent a module, a program segment, or a part of the code, and the module, the program segment, or the part of the code includes one or more executable instructions for implementing the specified logic function. It should also be noted that, in some alternative implementations, the functions marked in the blocks can also occur in a different order from the order marked in the drawings. For example, two blocks in connection can actually be executed substantially in parallel, or they can sometimes be executed in the reverse order, which depends on the functions involved. It should also be noted that each block in the block diagrams and/or flow diagrams, and the combination of the blocks in the block diagrams and/or flow diagrams, can be implemented by a dedicated hardware-based system that performs the specified functions or operations, or can be realized by a combination of dedicated hardware and computer instructions.

Generally speaking, the various examples and embodiments of the present disclosure can be implemented in hardware or special purpose circuits, software, firmware, logic, or any combination thereof. Certain aspects can be implemented in hardware, while other aspects can be implemented in firmware or software that may be executed by a controller, microprocessor, or other computing device. When various aspects of the embodiments of the present disclosure are illustrated or described as block diagrams, flow diagrams, or described by using some other graphical, it will be understood that the blocks, devices, systems, techniques, or methods described herein can be regarded as non-limiting examples implemented in hardware, software, firmware, dedicated circuits or logic, general-purpose hardware or controllers or other computing devices, or some combinations thereof.

The processor in the embodiments of the present disclosure can be an integrated circuit chip with signal processing capability. The above processor can be a general-purpose processor, a digital signal processor (DSP), an application specific integrated circuit (ASIC), a field programmable gate array (FPGA) or other programmable logic devices, discrete gate or transistor logic devices, or discrete hardware components, and the processor can implement or execute the methods, steps, and logical block diagrams disclosed in the embodiments of the present disclosure. The general-purpose processor can be a microprocessor or the processor can also be any conventional processor, etc., and can be of an X86 architecture or an ARM architecture.

The computer-readable storage medium in the embodiments of the present disclosure can be a volatile memory or a non-volatile memory, or can include both the volatile memory and the non-volatile memory. The non-volatile memory can be read only memory (ROM), programmable read only memory (PROM), erasable programmable read only memory (EPROM), electrically erasable programmable read only memory (EEPROM), or flash memory. The volatile memory can be random access memory (RAM), which acts as an external cache. By way of exemplary but not restrictive description, many forms of RAM are available, such as static random access memory (SRAM), dynamic random access memory (DRAM), synchronous dynamic random access memory (SDRAM), double data rate synchronous dynamic random access memory (DDRSDRAM), enhanced synchronous dynamic random access memory (ESDRAM), synchronous link dynamic random access memory (SLDRAM), and direct memory bus random access memory (DRRAM). It should be noted that the memories of the systems and methods described herein are intended to include, but are not limited to, these memories and any other suitable types of memories.

The exemplary embodiments of the present disclosure described in detail above are merely illustrative and not restrictive. Those skilled in the art should understand that various modifications, combinations or sub-combinations can be made to these embodiments without departing from the principle and spirit of the present disclosure, and such modifications should fall within the scope of the present disclosure.

1. A method for processing physiological data, comprising:
acquiring one or more physiological data of a detected object;
calculating a state judgment value corresponding to each of the one or more physiological data based at least in part on a basic assignment table; and
calculating health data of the detected object based at least in part on a weight assignment table and the state judgment value.

2. The method according to claim 1, wherein the one or more physiological data are acquired by a non-invasive vital sign detection device.

3. The method according to claim 1, wherein the basic assignment table comprises at least one of following assignment items corresponding to the one or more physiological data: a top value, a bottom value, an increase fragment value, and a decrease fragment value.

4. The method according to claim 1, wherein calculating the state judgment value corresponding to each of the one or more physiological data comprises:
in response to a value V of one physiological data PD of the one or more physiological data being greater than a top value RC corresponding to the one physiological data PD, and according to an increase fragment value α corresponding to the one physiological data PD, calculating a state judgment value F_{PD} corresponding to the one physiological data PD,
wherein
$${F}_{P D} = 1 - {2}^{\frac{V - R C}{α}} .$$

5. The method according to claim 1, wherein calculating the state judgment value corresponding to each of the one or more physiological data comprises:
in response to a value V of one physiological data PD of the one or more physiological data being less than a bottom value RF corresponding to the one physiological data PD, and according to a decrease fragment value β corresponding to the one physiological data PD, calculating a state judgment value F_{PD} corresponding to the one physiological data PD,
wherein
$${F}_{P D} = 1 - {2}^{\frac{R F - V}{β}} .$$

6. The method according to claim 1, wherein calculating the state judgment value corresponding to each of the one or more physiological data comprises:
in response to a value V of one physiological data PD of the one or more physiological data being greater than a bottom value RF corresponding to the one physiological data PD and less than a top value RC corresponding to the one physiological data PD, a state judgment value F_{PD} corresponding to the one physiological data PD being zero.

7. The method according to claim 1, wherein calculating health data of the detected object comprises:
according to a weight θ_{PD} corresponding to each of the one or more physiological data in the weight assignment table, calculating health data P of the detected object, wherein
P=S+Σ_{PD=1}ⁿF_{PD}*ω_{PD},
n represents a count of the one or more physiological data, F_{PD} represents a state judgment value corresponding to one physiological data PD of the one or more physiological data, and S represents a maximum value of the health data of the detected object.

8. The method according to claim 1, wherein in response to the health data of the detected object being associated with a cardiovascular state of the detected object, the one or more physiological data comprise at least one of following items: systolic blood pressure, diastolic blood pressure, peripheral pulse rate, heart beat volume, and blood viscosity.

9. The method according to claim 1, wherein in response to the health data of the detected object being associated with a pulmonary circulation function of the detected object, the one or more physiological data comprise at least one of following items: oxygen partial pressure, oxygen content, blood oxygen saturation, carbon dioxide partial pressure, total carbon dioxide, pH value, hemoglobin, red blood cell count, and hematocrit.

10. The method according to claim 3, wherein any one of the top value and the bottom value is associated with gender of the detected object.

11. The method according to claim 10, wherein calculating the state judgment value corresponding to each of the one or more physiological data comprises:
acquiring a bottom value and a top value of an assignment item corresponding to each of the one or more physiological data according to the gender of the detected object.

12. A device for processing physiological data, comprising:
an acquisition circuit, configured to acquire one or more physiological data of a detected object;
an area calculation circuit, configured to calculate a state judgment value corresponding to each of the one or more physiological data based at least in part on a basic assignment table; and
a comprehensive calculation circuit, configured to calculate health data of the detected object based at least in part on a weight assignment table and the state judgment value.

13. The device according to claim 12, further comprising:
a first memory, configured to store the basic assignment table; and
a second memory, configured to store the weight assignment table.

14. The device according to claim 12, wherein the acquisition circuit is provided in a non-invasive vital sign detection device.

15. The device according to claim 12, wherein the area calculation circuit is further configured to,
in response to a value of one physiological data of the one or more physiological data being greater than a top value corresponding to the one physiological data, and according to an increase fragment value corresponding to the one physiological data, calculate a state judgment value corresponding to the one physiological data,
wherein the state judgment value is nonlinearly inversely proportional to the value of the one physiological data and is nonlinearly proportional to the increase fragment value.

16. The device according to claim 12, wherein the area calculation circuit is further configured to,
in response to a value of one physiological data of the one or more physiological data being less than a bottom value corresponding to the one physiological data, and according to a decrease fragment value corresponding to the one physiological data, calculate a state judgment value corresponding to the one physiological data,
wherein the state judgment value is nonlinearly proportional to the value of the one physiological data and is nonlinearly proportional to the decrease fragment value.

17. The device according to claim 12, wherein the area calculation circuit is further configured to,
in response to a value of one physiological data of the one or more physiological data being greater than a bottom value corresponding to the one physiological data and less than a top value corresponding to the one physiological data, calculate a state judgment value corresponding to the one physiological data to be zero.

18. The device according to claim 12, wherein the comprehensive calculation circuit is further configured to,
according to a weight corresponding to each of the one or more physiological data in the weight assignment table, calculate the health data of the detected object.

19. A device for processing physiological data, comprising:
a processor, and
a memory, wherein the memory stores computer executable instructions, and the computer executable instructions are executed by the processor to perform the method for processing physiological data according to claim 1.

20. A non-volatile storage medium, storing computer executable instructions, wherein the computer executable instructions are executed by a processor to perform the method for processing physiological data according to claim 1.
